(19) United States
(12) Patent Application Publication
(10) Pub. No.: US 2005/0004549 A1
(43) Pub. Date: Jan. 6, 2005

(54) Disposable pant-like undergarment

(75) Inventors: Cynthia Dianne Maas, Appleton, WI (US); Judith Ann Musil, Appleton, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/612,641

(22) Filed: Jul. 1, 2003

(51) Int. Cl. A61F 13/15; A61F 13/20

(52) U.S. Cl. 604/385.29

(57) ABSTRACT

A disposable pant-like undergarment is disclosed for absorbing human discharge. The pant-like undergarment includes stretchable front and back panels. The front panel has first and second elastic bands secured along its first and second ends. The back panel has third and fourth elastic bands secured along its first and second ends. An absorbent assembly is secured to the front and back panels and is capable of being folded to enable the front panel to overlap the back panel. A pair of seams joins the front panel to the back panel to form an undergarment having a waist opening and a pair of leg openings. The first and third elastic bands create a waist band located adjacent to the waist opening and the second and fourth elastic bands create a pair of leg bands located adjacent to the pair of leg openings. The fourth elastic band has a stretching force that is greater than that of the second elastic band.

### BACKGROUND OF THE INVENTION

A disposable pant-like undergarment is designed for absorbing human exudate. The pant-like undergarment is similar in appearance, size and shape to a regular cloth underwear except that it is not designed to be laundered and reused two or more times. A disposable pant-like undergarment is intended to be worn by persons, including infants, toddlers, adolescents, or adults, and is designed for single or temporary use. The disposable pant-like undergarment is meant to be disposed of after being used once instead of being laundered or dry cleaned for re-use. Generally speaking, disposable pant-like undergarments have a “closed” construction like regular cloth underwear and are designed to be pulled up around the user's torso without having to first open the undergarment in order to place it on a person's body. The stretchability of the material used to construct the disposable pant-like undergarment permits the undergarment to snugly conform to the anatomy of the user's torso. The disposable pant-like undergarment can be manufactured to be an infant diaper, a child training pant, an adolescent garment, an adult incontinence garment, a feminine menstrual pant, etc.

Some disposable pant-like undergarments manufactured today resemble regular cloth underwear in that they have a waist opening and a pair of leg openings. The human skin in the waist/abdomen area tends to be relatively sensitive to irritation. Another benefit of forming the waist band on the outside of the undergarment is that it provides a raised area which the wearer can easily grasp when pulling the undergarment up and around their torso. To the contrary, the pair of leg bands can be situated on the inside of the undergarment since the skin is less susceptible to irritation around the thighs. In some instances, experimentation has also shown that by forming the leg bands around the inside of each leg opening, the undergarment performs better in preventing bodily fluids from leaking out around the thighs. However, some believe that the skin present in the front of the thighs tends to be more sensitive than the skin in the rear of the thighs. Experimentation has further shown that it is possible to manufacture an undergarment where the tension surrounding the pair of leg bands is varied. A lower tension in the front of the pair of leg bands is more comfortable to the wearer of the undergarment. It has now been discovered that by varying the elastic tension in the pair of the leg bands, one can obtain a snugger fit without the fear of forming red marks on the wearer's thighs. It has also been noted that the rise and pitch of the material forming the back panel of an undergarment is normally greater than the rise and pitch of the material forming the front panel. By increasing the elastic tension in the back panel of each of the pair of leg bands, an improved fit can be obtained which reduces the likelihood of fluid leakage.

Now a disposable pant-like undergarment for absorbing human discharges has been invented that includes a waist band located on an exterior surface and a pair of leg bands located on an interior surface. The tension between the front and back regions of each of the pair of leg bands is varied to reduce the occurrence of red marks forming on the user's thighs.

### SUMMARY OF THE INVENTION

Briefly, this invention relates to a disposable absorbent article for absorbing human discharge. The disposable pant-like undergarment includes stretchable front and back panels. The front panel has a first end, a second end, a first side edge, a second side edge, a first elastic band secured along the first end and a second elastic band secured along the second end. The back panel has a first end, a second end, a first side edge, a second side edge, a third elastic band secured along the first end and a fourth elastic band secured along the second end. An absorbent assembly including a liquid pervious bodyside liner, a liquid-impervious outer cover, and an absorbent positioned therebetween, is secured to the stretchable front and back panels. The absorbent assembly is capable of being folded to enable the front panel to overlap the back panel. A pair of seams joins the stretchable front and back panels together at the first and second side edges to form a pant-like undergarment having a waist opening and a pair of leg openings. The first and third elastic bands create a waist band located adjacent to the waist opening and the second and fourth elastic bands create a pair of leg bands located adjacent to the pair of leg openings, and the fourth elastic band has a stretching force that is greater than that of the second elastic band.

### BRIEF DESCRIPTION OF THE DRAWINGS

FIG. 1 is a perspective view of a disposable pant-like undergarment having a waist band formed about the waist opening and a pair of leg bands formed about the pair of leg openings and wherein the stretching force around each of the pair of leg bands varies.

FIG. 2 is a plane view of the disposable pant-like undergarment shown in FIG. 1 depicting an absorbent assembly secured to the inner surface of stretchable front and back panels and showing four elastic bands that make up the waist band and the pair of leg bands in the finished undergarment.

FIG. 3 is a side view of the disposable pant-like undergarment shown in FIG. 2 taken along line 3--3.

### DETAILED DESCRIPTION

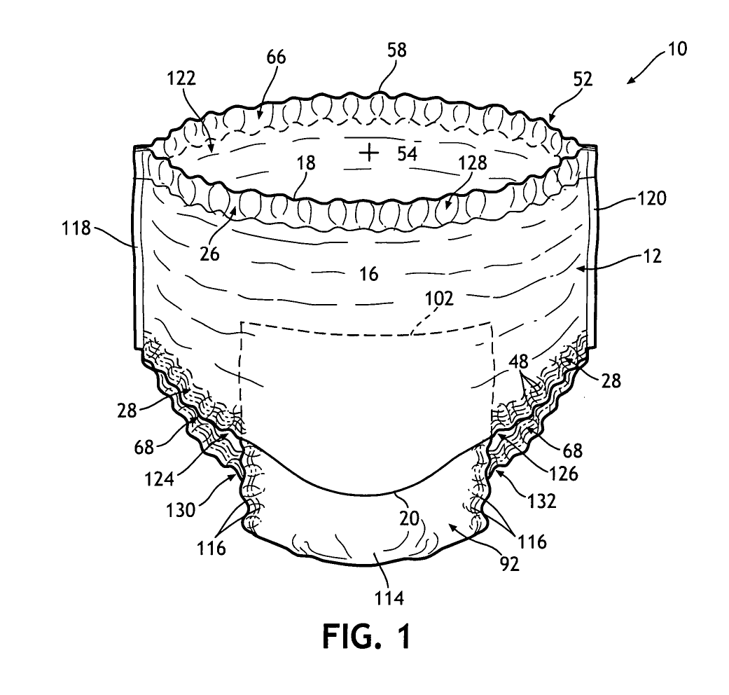

Referring to FIG. 1, a disposable pant-like absorbent undergarment 10 is depicted. The disposable pant-like undergarment 10 is intended to be worn by persons, including infants, toddlers, adolescents, or adults, and is designed for a single or temporary use. The disposable pant-like undergarment 10 is meant to be disposed of after being used once instead of being laundered or dry cleaned for re-use. Generally speaking, when a consumer or user encounters the disposable pant-like undergarment 10, it has a “closed” pant-like construction like conventional cloth underpants and is designed to be pulled up around the user's torso without having to first open the undergarment 10 in order to place it on a person's body. In FIG. 1, the disposable pant-like undergarment 10 is shown as it would appear just prior to being pulled up around a user's torso.

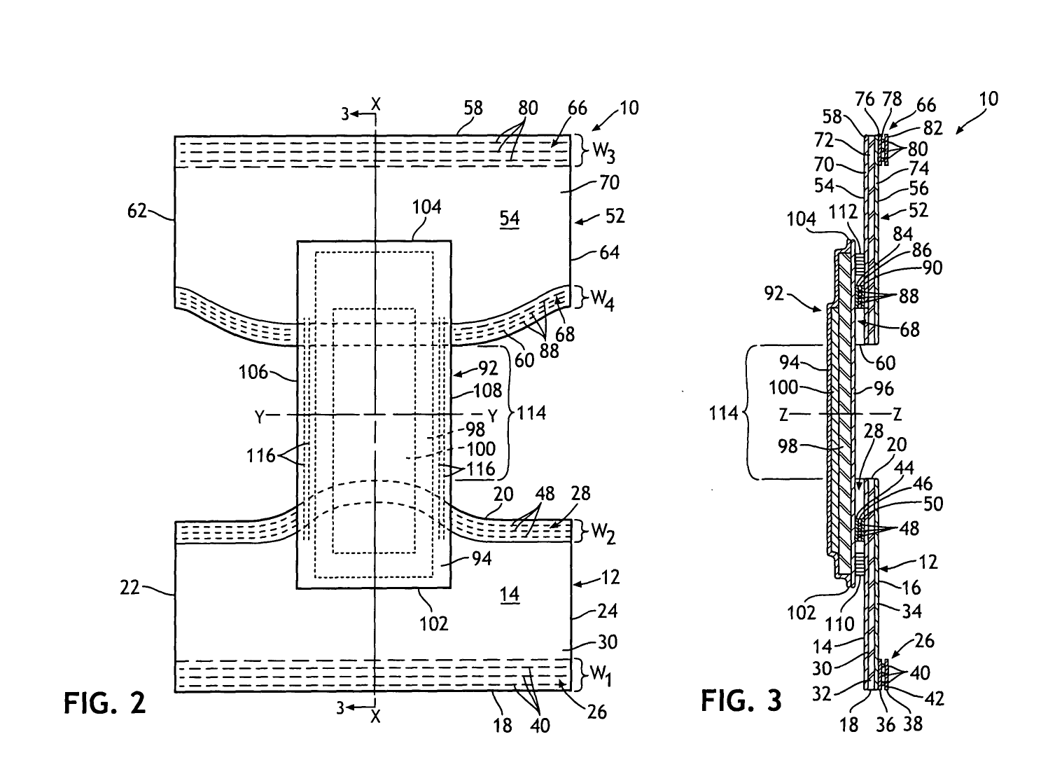

Referring to FIGS. 2 and 3, the disposable pant-like undergarment 10 is depicted in an open position solely for the purpose of better showing the various components. The open disposable pant-like undergarment 10 has a longitudinal central axis X--X, a transverse central axis Y--Y and a vertical central axis Z--Z. The disposable pant-like undergarment 10 includes a stretchable front panel 12 having an inner surface 14, an outer surface 16, a first end 18, a second end 20, a first side edge 22 and a second side edge 24. The front panel 12 also has a first elastic band 26 secured along the first end 18 and a second elastic band 28 secured along the second end 20. By “elastic” it is meant a material that is returning or capable of returning to an initial form or state after deformation. The terms “elastic” and “elastomeric” are used herein to mean any material which, upon application of a biasing force, is stretchable, that is, elongatable, to a stretched, biased length which is at least about 125 percent of its relaxed unbiased length, and which, will recover at least 40 percent of its elongation upon release of the stretching, elongating force. A hypothetical example would be a one (1) inch sample of material which is elongated to at least 1.25 inches and which, upon being elongated to 1.25 inches and released, will recover to a length of not more than 1.1 inches. Many elastic materials may be elongated by much more than 25 percent (i.e., much more than 125 percent of their relaxed length), for example, elongated 200 percent or more, and many of these will recover to substantially their initial relaxed length, for example, to within 105 percent of their initial relaxed length, upon release of the stretching force.

As used herein, the term “non-elastic” refers to any material which does not fall within the definition of “elastic” defined above.

The first elastic band 26 extends between the first and second side edges, 22 and 24 respectively. Desirably, the first elastic band 26 extends laterally across the width of the front panel 12 from the first side edge 22 to the second side edge 24. Likewise, the second elastic band 28 extends between the first and second side edges, 22 and 24 respectively. Desirably, the second elastic band 28 extends laterally across the width of the front panel 12 from the first side edge 22 to the second side edge 24. The first and second elastic bands, 26 and 28 respectively, can be linear or non-linear in configuration. As depicted in FIG. 2, the first elastic band 26 is linear and extends laterally across the front panel 12. The second elastic band 28 is non-linear in configuration to mirror the profile of the second end 20. The second elastic band 28 also extends laterally across the front panel 12 but in a circuitous fashion.

As stated above, the front panel 12 and the first and second bands, 26 and 28 respectively, are stretchable. By “stretchable” it is meant that each is formed from a material that is capable of being extended or elongated under a force and then is capable of contracting back to or towards its initial length once the force is removed. For example, say that the front panel 12 had a width of 10 inches (25.4 cm) measured parallel to the transverse axis Y--Y from the first side edge 22 to the second side edge 24, and a force is applied to cause the front panel 12 to elongate to 15 inches (38.1 cm) along the transverse axis Y--Y. Once the force used to stretch or elongate the front panel 12 is removed, the front panel 12 is allowed to contract or return back to or towards its original pre-stretched length of 10 inches (25.4 cm). The contracted length of the front panel 12 is equal to or approximately equal to the original pre-stretched distance, that being the distance between the first and second side edges, 26 and 28 respectively. An elastic material is stretchable and will exhibit the same characteristics as a rubber band that has an initial length before it is stretched or extended by applying a pulling force to opposite ends thereof. Once the force is removed, the rubber band will contract and return to its original pre-stretched length.

The front panel 12 can be formed from various stretchable materials. A desired material is an elastic material. The front panel 12 can also be formed from two or more layers wherein at least one of the layers is elastic. By “layer” it is meant a single sheet, a film, a net-like material or a plurality of elastic strands. When a plurality of elastic strands is utilized, they can be positioned on another layer to form a two layer structure. Alternatively, the plurality of elastic strands can be located between two or more layers to form a multilayered structure. The front panel 12 is formed such that essentially the entire panel is capable of being stretched and then will contract in at least one direction. Desirably, the front panel 12 is formed such that essentially the entire panel can stretch and contract in two or more directions. By “essentially the entire panel” is meant that about 90% to 100% of the front panel 12 is stretchable when a force is applied to it and is contractible when the force is removed. Desirably, the stretch and contraction of the front panel 12 is in a direction approximately parallel to the transverse axis Y--Y. The transverse direction extends laterally across the torso from one hip bone to the other hip bone. More desirably, the stretch and contraction of the front panel 12 is in at least two directions, one direction being approximately parallel to the longitudinal axis X--X and the other direction being approximately parallel to the transverse axis Y--Y. Most desirably, the stretch and contraction of the front panel 12 is in multiple directions, or stated another way, in three or more directions extending over an arc of 360 degrees. The ability of the front panel 12 to contract will provide a restrictive force during use of the disposable pant-like undergarment 10 to ensure that it snugly conforms to the anatomy of the wearer's torso.

The front panel 12 can be constructed from various stretchable materials having elastic characteristics. The front panel 12 can be formed from a single material or be a laminate. The laminate can include two or more layers. A good material for the front panel 12 is a stretch bonded laminate (SBL). Exemplary SBL materials are described in U.S. Pat. No. 4,720,415, which is hereby incorporated by reference and made a part hereof. The SBL can consist of three layers 30, 32 and 34. In a stretch bonded laminate, the elastic core or middle layer 32 is elongated before the two outer nonwoven layers 30 and 34 are attached. The attachment can be by an adhesive, by heat, by pressure, by a combination of heat and pressure, etc. Another material option for the front panel 12 is a necked bonded laminate (NBL). Exemplary NBL materials are described in U.S. Pat. No. 5,336,545, which is hereby incorporated by reference and made a part hereof. The NBL can consist of an elastic core or middle layer 32 that is not pre-stretched prior to being attached to the two outer nonwoven layers 30 and 34. The outer layers 30 and 34 are necked stretched before the elastic core or middle layer 32 is attached to them. Other examples of elastomeric materials that can be used for the front panel 12 include a continuous filament stretch bonded laminate (CFSBL), a vertical filament laminate (VFL), a necked stretch bonded laminate (NSBL) and a necked thermal laminate (NTL). Combinations of the above materials can also be used. Exemplary CFSBL materials are described in U.S. Pat. No. 5,385,775, which is hereby incorporated by reference and made a part hereof. Other elastic materials known to those skilled in the art can also be used.

It should be noted that the front panel 12 can be constructed from an elastic film that is capable of being stretched in at least one direction and desirably in both the machine direction and the cross-direction. Alternatively, the front panel 12 can be an elastic nonwoven that has a machine direction stretch or a cross-direction stretch. Extensible materials can also be used to form the front panel 12 of the disposable pant-like undergarment 10.

Alternatively, the front panel 12 can be formed from two outer layers 30 and 34 with a plurality of elastic strands sandwiched therebetween. The elastic strands can be formed from LYCRA®). LYCRA® is a registered trademark of E.I. Du Pont De Nemours & Co., having an office at 1007 Market Street, Wilmington, Del. 19898. The elastic strands can be aligned approximately parallel to one another or be angled or skewed relative to one another. The elastic strands can also be uniformly or randomly spaced apart from one another. The elastic strands can vary in shape, size, configuration, and/or length. The diameter and/or cross-sectional configuration of the elastic strands, the decitex (weight in grams per 10,000 meters of a strand) of the elastic strands, and the tension imparted into the elastic strands can all be varied to suit one's particular product needs. The elastic strands can have a round, semi-circular, square, rectangular, oval or some other geometrical configuration. The elastic strands can overlap, intersect or crisscross at least one other elastic strand, if desired. The various ways of positioning, orienting, and adhering the elastic strands to the two outer layers 30 and 34 are well known to those skilled in the art.

Referring again to FIG. 3, the first and second elastic bands, 26 and 28 respectively, can be formed from the same material as was used to make the front panel 12 or they can be formed from different materials. The first elastic band 26 is depicted as including a first outer layer 36, a second or middle layer 38 formed from a plurality of elastic strands 40, and a third layer 42. The elastic strands 40 making up the second layer 38 are sandwiched between the first and third outer layers, 36 and 42 respectively. The number of elastic strands 40 in the second or middle layer 38 can vary from 1 to about 50. Desirable, the number of elastic strands 40 in the second or middle layer 38 can vary from about 2 to about 35. More desirably, the number of elastic strands 40 in the second or middle layer 38 can vary from about 3 to about 20 strands. The shape, size, cross-sectional configuration, length, decitex, tension imparted into each elastic strand 40, etc. can vary to suit one's particular needs. These characteristics as well as the way the elastic strands 40 are positioned, oriented and adhered to the two outer layers 36 and 42 are well known to those skilled in the art.

The second elastic band 28 is depicted as being a three layer laminate similar to the first elastic band 26. The second elastic band 28 includes a first outer layer 44, a second or middle layer 46 formed from a plurality of elastic strands 48, and a third layer 50. The elastic strands 48 making up the second layer 46 are sandwiched between the first and third outer layers, 44 and 50 respectively. The number of elastic strands 48 in the second or middle layer 46 can vary from 1 to about 20. Desirable, the number of elastic strands 48 in the second or middle layer 46 can vary from about 2 to about 10. More desirably, the number of elastic strands 48 in the second or middle layer 46 can vary from about 3 to about 5 strands. The shape, size, cross-sectional configuration, length, decitex, tension imparted into each elastic strand 48, etc. can vary to suit one's particular needs. These characteristics, as well as the way the elastic strands 48 are positioned, oriented and adhered to the two outer layers 44 and 50, are well known to those skilled in the art.

The first elastic band 26 is secured to the outer surface 16 of the front panel 12 approximate the first end 18. Various means for securing or attaching the first elastic band 26 to the front panel 12 can be used. The attachment can be by an adhesive, by ultrasonics, by heat, by pressure, by a combination of heat and pressure, by mechanical means such as sewing with thread, etc. The first elastic band 26 can have a width (w₁) measured parallel to the longitudinal axis X--X of from between about 0.25 inches to about 2 inches (about 6.4 mm to about 51 mm). Desirably, the width (w₁) of the first elastic band 26 is from between about 0.375 inches to about 1.5 inches (about 9.5 mm to about 38 mm). More desirably, the width (w₁) of the first elastic band 26 is from between about 0.5 inches to about 1 inch (about 12 mm to about 25 mm). The length of the first elastic band 26 can extend completely across the front panel 12 from the first side edge 22 to the second side edge 24. Alternatively, the first elastic band 26 can extend across only a portion of the distance between the first and second side edges, 22 and 24 respectively. For example, the first elastic band 26 may terminate short of the first and second side edges, 22 and 24 respectively. Desirably, the first elastic band 26 will extend across at least about 75% of the width of the front panel 12. More desirably, the first elastic band 26 will extend across at least about 90% of the width of the front panel 12. Even more desirably, the first elastic band 26 will extend across at least about 95% of the width of the front panel 12. Most desirably, the first elastic band 26 will extend completely across the width of the front panel 12.

The second elastic band 28 is secured to the inner surface 14 of the front panel 12 approximate the second end 20. Various means for securing or attaching the second elastic band 28 to the front panel 12 can be used. The attachment can be by an adhesive, by ultrasonics, by heat, by pressure, by a combination of heat and pressure, by mechanical means such as sewing with thread, etc. The second elastic band 28 can have a width (w₂) measured parallel to the longitudinal axis X--X of from between about 0.25 inches to about 1 inch (about 6.4 mm to about 25 mm). Desirably, the width (w₂) of the second elastic band 28 is from between about 0.375 inches to about 0.75 inches (about 9.5 mm to about 19 mm). More desirably, the width (W₂) of the second elastic band 28 is from between about 0.5 inches to about 0.625 inches (about 12 mm to about 15.8 mm). The length of the second elastic band 28 can extend completely across the front panel 12 from the first side edge 22 to the second side edge 24. Alternatively, the second elastic band 28 can extend across only a portion of the distance between the first and second side edges, 22 and 24 respectively. For example, the second elastic band 28 may terminate short of the first and second side edges, 22 and 24 respectively. Desirably, the second elastic band 28 will extend across at least about 75% of the width of the front panel 12. More desirably, the second elastic band 28 will extend across at least about 90% of the width of the front panel 12. Even more desirably, the second elastic band 28 will extend across at least about 95% of the width of the front panel 12. Most desirably, the second elastic band 28 will extend completely across the width of the front panel 12.

Referring again to FIGS. 2 and 3, the disposable pant-like undergarment 10 also includes a stretchable back panel 52 having an inner surface 54, an outer surface 56, a first end 58, a second end 60, a first side edge 62 and a second side edge 64. The back panel 52 has a third elastic band 66 secured along the first end 58 and a fourth elastic band 68 secured along the second end 60. The third elastic band 66 extends between the first and second side edges, 62 and 64 respectively. Desirably, the third elastic band 66 extends laterally across the width of the back panel 52 from the first side edge 62 to the second side edge 64. Likewise, the fourth elastic band 68 extends between the first and second side edges, 62 and 64 respectively. Desirably, the fourth elastic band 68 extends laterally across the width of the back panel 52 from the first side edge 62 to the second side edge 64. The third and fourth elastic bands, 66 and 68 respectively, can be linear or non-linear in configuration. As depicted in FIG. 2, the third elastic band 66 is linear and extends laterally across the back panel 52. The fourth elastic band 68 is arcuate in configuration to mirror the profile of the second end 60. The fourth elastic band 68 also extends laterally across the back panel 52 but in a circuitous fashion.

As stated above, the back panel 52 is stretchable. The term “stretchable” has been defined above. The back panel 52 can be formed from a stretchable material that is elastic. The back panel 52 can also be formed from two or more layers wherein at least one of the layers is elastic. By “layer” it is meant a single sheet, a film, a net-like material or a plurality of elastic strands. When a plurality of elastic strands is utilized, they can be positioned on another layer to form a two-layer structure. Alternatively, the plurality of elastic strands can be located between two or more outer layers to form a multilayered structure. The back panel 52 is formed such that essentially the entire panel is capable of being stretched and contracted in at least one direction, and desirably, in two or more directions. By “essentially the entire panel” is meant that about 90% to 100% of the back panel 52 is stretchable when a force is applied to it and is contractible when the force is removed. The stretch and contraction can be in one direction or in two or more directions. Desirably, the stretch and contraction of the back panel 52 is in a direction approximately parallel to the transverse axis Y--Y. The transverse direction extends laterally across the torso from one hip bone to the other hip bone. More desirably, the stretch and contraction of the back panels 52 is in at least two directions, one direction being approximately parallel to the longitudinal axis X--X and the other direction being approximately parallel to the transverse axis Y--Y. Most desirably, the stretch and contraction of the back panel 52 is in multiple directions, or stated another way, in three or more directions extending over an arc of 360 degrees. The ability of the back panel 52 to contract will provide a restrictive force during use of the disposable pant-like undergarment 10 to ensure that it snugly conforms to the anatomy of the wearer's torso.

The back panel 52 can be constructed from various stretchable materials having elastic characteristics. The back panel 52 can be formed from a single material or be a laminate. The laminate can include two or more layers. A good material for the back panel 52 is a stretch bonded laminate (SBL). Exemplary SBL materials have been described earlier. The SBL can consist of three layers 70, 72 and 74. In a stretch bonded laminate, the elastic core or middle layer 72 is elongated before the two outer nonwoven layers 70 and 74 are attached. The attachment can be by an adhesive, by heat, by pressure, by a combination of heat and pressure, etc. Another material option for the back panel 52 is a necked bonded laminate (NBL). Exemplary SBL materials have been described earlier. In a NBL, the elastic core or middle layer 72 is not pre-stretched prior to being attached to the two outer nonwoven layers 70 and 74. The outer layers 70 and 74 are necked stretched before the elastic core or middle layer 72 is attached to them. Other examples of elastomeric materials that can be used for the back panel 52 include a continuous filament stretch bonded laminate (CFSBL), a vertical filament laminate (VFL), a necked stretch bonded laminate (NSBL) and a necked thermal laminate (NTL). Combinations of the above materials can also be used. Exemplary CFSBL materials have been described earlier. Other elastic materials known to those skilled in the art can also be used.

It should be noted that the back panel 52 can be constructed from an elastic film that is capable of being stretched in at least one direction and desirably in both the machine direction and the cross-direction. Alternatively, the back panel 52 can be an elastic nonwoven that has a machine direction stretch or a cross-direction stretch. Extensible materials can also be used to form the back panel 52 of the disposable pant-like undergarment 10.

Alternatively, the back panel 52 can be formed from two outer layers 70 and 74 with a plurality of elastic strands sandwiched therebetween. The elastic strands can be formed from LYCRA®. LYCRA® is a registered trademark of E.I. Du Pont De Nemours & Co., having an office at 1007 Market Street, Wilmington, Del. 19898. The elastic strands can be aligned approximately parallel to one another or be angled or skewed relative to one another. The elastic strands can also be uniformly or randomly spaced apart from one another. The elastic strands can vary in shape, size, configuration, and/or length. The diameter and/or cross-sectional configuration of the elastic strands, the decitex (weight in grams per 10,000 meters of a strand) of the elastic strands, and the tension imparted into the elastic strands can all be varied to suit one's particular product needs. The elastic strands can have a round, semi-circular, square, rectangular, oval or some other geometrical configuration. The elastic strands can overlap, intersect or crisscross at least one other elastic strand. The various ways of positioning, orienting, and adhering the elastic strands to the two layers 30 and 34 are well known to those skilled in the art.

Referring again to FIG. 3, the third and fourth elastic bands, 66 and 68 respectively, can be formed from the same material as was used to make the back panel 52 or they can be formed from different materials. The third elastic band 66 is depicted as including a first outer layer 76, a second or middle layer 78 formed from a plurality of elastic strands 80, and a third layer 82. The elastic strands 80 making up the second layer 78 are sandwiched between the first and third outer layers, 76 and 82 respectively. The number of elastic strands 80 in the second or middle layer 78 can vary from 1 to about 50. Desirable, the number of elastic strands 80 in the second or middle layer 78 can vary from about 2 to about 35. More desirably, the number of elastic strands 80 in the second or middle layer 78 can vary from about 3 to about 20 strands. The shape, size, cross-sectional configuration, length, decitex, tension imparted into each elastic strand 80, etc. can vary to suit one's particular needs. These characteristics, as well as the way the elastic strands 80 are positioned, oriented and adhered to the two outer layers 76 and 82 are well known to those skilled in the art.

The fourth elastic band 68 is depicted as being a three layer laminate similar to the third elastic band 66. The second elastic band 68 includes a first outer layer 84, a second or middle layer 86 formed from a plurality of elastic strands 88, and a third layer 90. The elastic strands 88 making up the second layer 86 are sandwiched between the first and third outer layers, 84 and 90 respectively. The number of elastic strands 88 in the second or middle layer 86 can vary from 1 to about 20. Desirable, the number of elastic strands 88 in the second or middle layer 86 can vary from about 2 to about 10. More desirably, the number of elastic strands 88 in the second or middle layer 86 can vary from about 3 to about 5 strands. The shape, size, cross-sectional configuration, length, decitex, tension imparted into each elastic strand 88, etc. can vary to suit one's particular needs. These characteristics as well as the way the elastic strands 88 are positioned, oriented and adhered to the two outer layers 84 and 90 are well known to those skilled in the art.

The third elastic band 66 is secured to the outer surface 56 of the back panel 52 approximate the first end 58. Various means for securing or attaching the third elastic band 66 to the back panel 52 can be used. The attachment can be by an adhesive, by ultrasonics, by heat, by pressure, by a combination of heat and pressure, by mechanical means such as sewing with thread, etc. The third elastic band 66 can have a width (w₃) measured parallel to the longitudinal axis X--X of from between about 0.25 inches to about 2 inches (about 6.4 mm to about 51 mm). Desirably, the width (w₃) of the third elastic band 66 is from between about 0.375 inches to about 1.5 inches (about 9.5 mm to about 38 mm). More desirably, the width (w₃) of the third elastic band 66 is from between about 0.5 inches to about 1 inch (about 12 mm to about 25 mm). The length of the third elastic band 66 can extend completely across the back panel 52 from the first side edge 62 to the second side edge 64. Alternatively, the third elastic band 66 can extend across only a portion of the distance between the first and second side edges, 62 and 64 respectively. For example, the third elastic band 66 may terminate short of the first and second side edges, 62 and 64 respectively. Desirably, the third elastic band 66 will extend across at least about 75% of the width of the back panel 52. More desirably, the third elastic band 66 will extend across at least about 90% of the width of the back panel 52. Even more desirably, the third elastic band 66 will extend across at least about 95% of the width of the back panel 52. Most desirably, the third elastic band 66 will extend completely across the width of the back panel 52.

The fourth elastic band 68 is secured to the inner surface 54 of the back panel 52 approximate the second end 60. Various means for securing or attaching the fourth elastic band 68 to the back panel 52 can be used. The attachment can be by an adhesive, by ultrasonics, by heat, by pressure, by a combination of heat and pressure, by mechanical means such as sewing with thread, etc. The fourth elastic band 68 can have a width (w₄) measured parallel to the longitudinal axis X--X of from between about 0.25 inches to about 1 inch (about 6.4 mm to about 25 mm). Desirably, the width (w₄) of the fourth elastic band 68 is from between about 0.375 inches to about 0.75 inches (about 9.5 mm to about 19 mm). More desirably, the width (w₄) of the fourth elastic band 68 is from between about 0.5 inches to about 0.625 inches (about 12 mm to about 15.8 mm). The length of the fourth elastic band 68 can extend completely across the back panel 52 from the first side edge 62 to the second side edge 64. Alternatively, the fourth elastic band 68 can extend across only a portion of the distance between the first and second side edges, 62 and 64 respectively. For example, the fourth elastic band 68 may terminate short of the first and second side edges, 62 and 64 respectively. Desirably, the fourth elastic band 68 will extend across at least about 75% of the width of the back panel 52. More desirably, the fourth elastic band 68 will extend across at least about 90% of the width of the back panel 52. Even more desirably, the fourth elastic band 68 will extend across at least about 95% of the width of the back panel 52. Most desirably, the fourth elastic band 68 will extend completely across the width of the back panel 52.

The front and back panels, 12 and 52 respectively, are discontinuous from one another in the longitudinal direction X--X. The inner surfaces 14 and 54 of the front and back panels, 12 and 52 respectively, are in direct contact with the user's skin and are also referred to as the bodyside surfaces. The outer surfaces 16 and 56 are situated opposite to the inner surfaces 14 and 54 and are spaced away from the user's skin. The outer surfaces 16 and 56 are also referred to as the garment facing surfaces since they can be in direct contact with the inner surfaces of the user's outer clothing.

Referring again to FIGS. 1-3, the disposable pant-like undergarment 10 also includes an absorbent assembly 92. The absorbent assembly 92 includes a liquid pervious bodyside liner 94, a liquid-impervious outer cover 96, and an absorbent 98 positioned therebetween. A surge layer 100 can be optionally used, which is located between the bodyside liner 94 and the absorbent 98. The surge layer 100 can function to rapidly acquire and temporarily retain body fluid, such as urine, before it can be absorbed into the absorbent 98. Desirably, the surge layer 100 is also capable of wicking body fluid lengthwise and/or widthwise across its surface as well as directing the body fluid downward in a z-direction, toward the absorbent 98.

Referring again to FIGS. 2 and 3, the absorbent assembly 92 has a first end 102, a second end 104, a first side edge 106 and a second side edge 108. The absorbent assembly 92 is shown secured to the inner surface 14 of the front panel 12 approximate the first end 102 by an attachment 110. The absorbent assembly 92 is also secured to the inner surface 54 of the back panel 52 approximate the second end 104 by an attachment 112. Alternatively, the absorbent assembly 92 can be secured to the outer surfaces 16 and 56 of the front and back panels, 12 and 52 respectively, if desired. The absorbent assembly 92 can be secured to the front and back panels, 12 and 52 respectively, after each panel has been stretched a predetermined amount. By attaching the absorbent assembly 92 to the inner surfaces 14 and 54 of the front and back panels, 12 and 52 respectively, the absorbent assembly 92 is capable of being in direct contact with the wearer's body.

The attachments 110 and 112 can be by various means and can include permanent attachments as well as removable or releasable attachments. Desirably, the attachments 110 and 112 are permanent attachments where they are not designed to be removed without destroying the bond. The attachments 110 and 112 can be formed by using glue, adhesive, ultrasonic bonds, thermal bonds, heat bonds, pressure bonds, heat and pressure bonds, or any other attachment mechanism known to those skilled in the art. The attachments 110 and 112 can also include a mechanical fastener, such as by sewing with thread, using buttons and button holes, using snaps, by employing hook and loop fasteners, etc. A hook and loop fastener is generally considered a releasable attachment. One type of hook and loop fastener is VELCRO® wherein a hook material is releasably engaged into a loop material. VELCRO® is a registered trademark of Velcro USA, Inc. having an office at 406 Brown Avenue, Manchester, N.H. 03103.

The attachments 110 and 112 can be formed along a continuous line or over a surface area having a predetermined length and width. Alternatively, the attachments 110 and 112 can consist of intermittent point bonds that are spaced apart from one another. For example, the intermittent point bonds can be formed by using a hot or cold melt adhesive or by forming ultrasonic bonds. Various bond formations can be used which are known to those skilled in the art. Desirably, the attachments 110 and 112 are formed using intermittent bonds because it allows the stretchable material forming the front and back panels, 12 and 52 respectively, to gather the absorbent assembly 92 as the elastic contracts. This gathering feature causes the absorbent assembly 92 to remain in direct contact with the user's body prior to and during the time period that the absorbent assembly 92 is being insulted with body fluid.

The front and back panels, 12 and 52 respectively, can be stretched in a direction approximately parallel to the transverse axis Y--Y, or in any other direction or directions, before the absorbent assembly 92 is secured to it. The amount of stretch can vary. No stretch is required if one does not wish to do so. Desirably, the front and back panels, 12 and 52 respectively, are stretched at least about 5% from a relaxed state before the absorbent assembly 92 is secured thereto. Desirably, the front and back panels, 12 and 52 respectively, are stretched at least about 10% from a relaxed state before the absorbent assembly 92 is secured thereto. More desirably, the front and back panels, 12 and 52 respectively, are stretched at least about 25% from a relaxed state before the absorbent assembly 92 is secured thereto. Most desirably, the front and back panels, 12 and 52 respectively, are stretched from between about 25% to about 1,000% from a relaxed state before the absorbent assembly 92 is secured thereto. The front and back panels, 12 and 52 respectively, extend laterally beyond the first and second side edges, 106 and 108 respectively, of the absorbent assembly 92. The front panel 12 also extends longitudinally beyond the first end 102 of the absorbent assembly 92 and the back panel 52 extends longitudinally beyond the second end 104 of the absorbent assembly 92. This overall size of the front and back panels, 12 and 52 respectively, allow them to elastically conform to the torso of the wearer.

Still referring to FIG. 2, when the absorbent assembly 92 is secured to the front and back panels, 12 and 52 respectively, a crotch region 114 is formed. The crotch region 114 separates the front panel 12 from the back panel 52 and is designed to cover the perineum area of the wearer. The crotch region 114 can cover a distance of a few inches in an infant diaper to several inches in an adult incontinence garment. For example, a crotch region 114 in an infant diaper may range from about 2 inches (about 5 centimeters (cm)) to about 10 inches (about 25 cm), while in an adult incontinence garment; the crotch region 114 may range from about 6 inches (about 15 cm) to about 20 inches (about 51 cm).

The absorbent assembly 92 can be stretchable or non-stretchable in relation to the front and back panels, 12 and 52 respectively. Desirably, the absorbent assembly 92 is non-stretchable in relation to the front and back panels, 12 and 52 respectively. By having the absorbent assembly 92 be non-stretchable in relation to the front and back panels, 12 and 52 respectively, it is meant that the absorbent assembly 92 will not stretch appreciably in the longitudinal or transverse directions. The reason for this is that the front and back panels, 12 and 52 respectively, are stretchable and can extend and contract to snugly conform to the user's anatomy, especially to his or her torso. Alternatively, the absorbent assembly 92 could be constructed with a pleated or folded construction, so as to be capable of being expanded in the longitudinal or transverse directions, if expansion of the absorbent assembly 92 is needed. The pleating or folding should occur before the absorbent assembly 92 is secured to the front and back panels, 12 and 52 respectively.

Normally, there is no need to have the absorbent assembly 92 gather as the front and back panels, 12 and 52 respectively, contract. However, the absorbent assembly 92 can be constructed and attached to the front and back panels, 12 and 52 respectively, in a way that will allow the absorbent assembly 92 to be gathered as the front and back panels, 12 and 52 respectively, contract in the transverse direction. In either circumstance, the absorbent assembly 92 should remain over the perineum. As the absorbent assembly 92 receives body fluid and/or excrement discharged by the wearer, it will be displaced outward, away from the user's torso. The attachments 110 and 112 assure that the absorbent assembly 92 covers the perineum but is capable of moving outward away from the torso as additional body fluid is received and retained.

Still referring to FIGS. 2 and 3, the disposable pant-like undergarment 10 also has at least one elastic member 116 positioned adjacent to and aligned approximately parallel to each of the first and second side edges 106 and 108 of the absorbent assembly 92. Each of the elastic members 116 is situated between the bodyside liner 94 and the outer cover 96. Each of the elastic members 116 provides a gasket to hold the first and second side edges, 106 and 108 respectively, of the absorbent assembly 92 against the user's body. Each of the elastic members 116 can be in the form of an elastic strand, ribbon or strip. Desirably, there are from 2 to 6 elastic members 116 positioned adjacent to each of the first and second side edges, 106 and 108 respectively. In FIGS. 2 and 3, two elastic members 116 are shown positioned adjacent to each of the first and second side edges, 106 and 108 respectively. The elastic members 116 can have a cross-sectional configuration that is round, square, rectangular or any other desired geometrical configuration. The elastic members 116 can be aligned parallel to the longitudinal axis X--X and should extend completely through the crotch region 114. Desirably, the ends of the elastic members 116 will be located within the front and back panels, 12 and 52 respectively.

Referring again to FIGS. 1 and 2, the absorbent assembly 92 is capable of being folded transversely, approximate the transverse axis Y--Y. The folding enables the front panel 12 to overlap the back panel 52. When the absorbent assembly 92 is folded, the first and second side edges 22 and 24 of the front panel 12 will align with the first and second side edges 62 and 64 of the back panel 52. After being folded, a pair of seams 118 and 120 is formed to join the front panel 12 to the back panel 52. The seam 118 secures the first side edge 22 of the front panel 12 to the first side edge 62 of the back panel 52. The other seam 120 secures the second side edge 24 of the front panel 12 to the second side edge 64 of the back panel 52. After the pair of seams 118 and 120 is formed, the disposable pant-like undergarment 10 has a waist opening 122 and a pair of leg openings 124 and 126. Since the front and back panels, 12 and 52 respectively, are formed from a stretchable material, the pair of leg openings 124 and 126 can expand or contract in size to accommodate the anatomy of the user.

Referring again to FIG. 1, one can clearly see that the first and third elastic bands, 26 and 66 respectively, cooperate to form a waist band 128. The waist band 128 is located adjacent to the waist opening 122. The waist band 128 can extend completely around the circumference of the waist opening 122 or extend over a portion of the circumference of the waist opening 122. Desirably, the waist band 128 will extend from between about 50% to about 100% of the circumference of the waist opening 122. More desirably, the waist band 128 will extend from between about 75% to about 100% of the circumference of the waist opening 122. Even more desirably, the waist band 128 will extend from between about 80% to about 100% of the circumference of the waist opening 122. Because of the pair of side seams 118 and 120, the waist band may not extend completely around the circumference of the waist opening 122.

It should be noted that the waist band 128 contains a number of elastic strands 40 and 80. The exact number of elastic strands 40 and 80 can vary and the number of elastic strands 40 can be less than, equal to or greater than the number of elastic strands 80. Likewise, the number of elastic strands 40 and 80 formed in the waist band 128 can be less than, equal to or greater than the number of elastic strands 48 and 88 present in the leg bands 130 and 132. Desirably, the waist band 128 contains at least twice as many elastic strands 40 and 80 as each of the pair of leg bands 130 and 132. More desirably, the waist band 128 contains a number of elastic strands 40 and 80 and each of the pair of leg bands 130 and 132 will contain a lesser number of elastic strands 48 and 88.

It should also be noted that the waist band 128 can have a width that is less than, equal to or be greater than the width of each of the leg bands 130 and 132. The width of the waist band 128 is measured parallel to the longitudinal axis X--X. Desirably, the width (w₁) of the first elastic band 26 and the width (w₃) of the third elastic band 66 are approximately equal in dimension. The width of the waist band 128 will then be equal to (w₁) or (w₃). Likewise, the width of the waist band 128 (w₁) or (w₃) can be less than, equal to or be greater than the width (w₂) or (w₄) of each of the pair of leg bands 130 or 132. Most desirably, the width of each of the leg bands 130 and 132 is equal.

It has been found by constructing a number of prototype disposable pant-like undergarments that good results can be obtained for an adult incontinent undergarment when the waist band 128 contains at least one elastic strand 40 and 80. Desirably, at least two elastic strands 40 and 80 will be present in the waist band 128. More desirably, at least three elastic strands 40 and 80 will be present in the waist band 128. When two or more elastic strands 40 and 80 are present, they can be uniformly spaced apart from one another. Experimentation has also shown that good results can be obtained for an adult incontinent undergarment when each of the pair of leg bands 130 and 132 contain at least one elastic strand 48 and 88. Desirably, at least two elastic strands 48 and 88 will be present in each of the leg bands 130 and 132. More desirably, at least three elastic strands 48 and 88 will be present in each of the leg bands 130 and 132. When two or more elastic strands 48 and 88 are present, they can be uniformly spaced apart from one another. It should be noted that the waist band 128 can contain the same number of elastic strands as are present in each of the leg bands 130 and 132. Alternatively, the waist band 128 can contain a greater or lesser number of elastic strands as are present in each of the leg bands 130 and 132.

The waist band 128 contains a number of elastic strands 40 and 80 and each is capable of being stretched a predetermined amount from an initial length. Desirably, each elastic strand 40 and 80 should be capable of being stretched at least 40% from an initial length. More desirably, each elastic strand 40 and 80 should be capable of being stretched at least 80% from an initial length. Even more desirably, each elastic strand 40 and 80 should be capable of being stretched at least 100% from an initial length. Still more desirably, each elastic strand 40 and 80 should be capable of being stretched at least 200% from an initial length. Most desirably, each elastic strand 40 and 80 should be capable of being stretched at least 500% from an initial length. Each of the pair of leg bands 130 and 132 contains a number of elastic strands 48 and 88 and each is capable of being stretched a predetermined amount from an initial length. Desirably, each elastic strand 48 and 88 should be capable of being stretched at least 40% from an initial length. More desirably, each elastic strand 48 and 88 should be capable of being stretched at least 80% from an initial length. Even more desirably, each elastic strand 48 and 88 should be capable of being stretched at least 100% from an initial length. Still more desirably, each elastic strand 40 and 80 should be capable of being stretched at least 200% from an initial length. Most desirably, each elastic strand 48 and 88 should be capable of being stretched at least 500% from an initial length.

The second and fourth elastic bands, 28 and 68 respectively, cooperate to form a pair of leg bands 130 and 132. Each of the pair of leg bands 130 and 132 is located adjacent to one of the pair of leg openings 124 and 126. The fourth elastic band 68 has a stretching force that is greater than that of the second elastic band 28. Desirably, the fourth elastic band 68 also has a contraction force that is greater than that of the second elastic band 28. The amount of force (in grams) required to stretch and contract the second and fourth elastic bands, 28 and 68 respectively, can be calculated using a multi-cycle stress/strain test.

### Test Procedure

A constant-rate-of-extension tensile tester with a computer-based acquisition and frame control system can be used. For the type of tensile tester being used, a load cell is selected wherein a majority of the peak load results fall between 10% and 90% of the capacity of the load cell. A tensile tester and load cell can be obtained from Instron Corporation having an office in Canton, Mass. 02021 or from MTS Systems Corporation having an office in Research Triangle Park, N.C. 27709-4226. The load cell can be conditioned (warmed up) prior to testing according to the manufacturer's specifications. The load cell should also be calibrated following the instruction manual.

The test should be conducted in a laboratory maintained at a conditioned testing environment of 23±2° C. and 50±5% relative humidity. Five test samples should be tested. Each test sample should be prepared after ambient conditions have been met. Each test sample should be cut to 25±1 mm in the non-extensible direction and 102±1 mm in the extensible direction. The five test samples will yield five test values that should be added and divided by 5 to obtain an average test value. The average test value of the stretching force and the average test value of the contraction force can then be compared to the claim limitations.

The following tensile test parameters should be followed:
1. The crosshead speed should be equal to 508±10 mm/minute;
2. The gage length of the raw material or composite should be equal to 76±1 mm;
3. Load units=grams-force;
4. Cycle elongation=80% strain;
5. Elongation data points can be at 10, 20, 30, 40, 50, 60, 70 and 80%;
6. Number of cycles=1.

Each of the test samples should be labeled with appropriate identification. Each test sample is independently and sequentially loaded into the pair of grips of the tensile tester. 76±1 mm of test sample should be present between the pair of grips. One should ensure that the preload does not exceed 10 grams-force in order to make sure that any slack in the test sample is removed. The tensile tester is then started and the pair of grips is moved apart at the designated crosshead speed. The extension (stretch) and contraction values are recorded. When the test is finished and the pair of grips has returned to there initial position, the grips are opened and the test sample is removed. The tensile tester is then readied to receive another test sample and the continuous testing is repeated until all the test samples have been tested.

For the present invention, the stretching force of the fourth elastic band 68 is greater than that of the second elastic band 28. In addition, the contraction force of the fourth elastic band 68 is greater than that of the second elastic band 28. The stretching and contraction force values will be compared at a predetermined extension. For example, when the test sample has been elongated to an extension of 40% and then contracted to 40%.

Desirably, the stretching force of the fourth elastic band 68 is at least 1.2 times greater than that of the second elastic band 28. More desirably, the stretching force of the fourth elastic band 68 is at least 1.5 times greater than that of the second elastic band 28. Most desirably, the stretching force of the fourth elastic band 68 is at least 2.0 times greater than that of the second elastic band 28. Likewise, the contraction force of the fourth elastic band 68 is at least 1.2 times greater than that of the second elastic band 28. More desirably, the contraction force of the fourth elastic band 68 is at least 1.5 times greater than that of the second elastic band 28. Most desirably, the contraction force of the fourth elastic band 68 is at least 2.0 times greater than that of the second elastic band 28. This means that the force needed to stretch or extend the fourth elastic band 68, located on the back of each of the pair of leg openings 124 and 126, is greater than the force needed to stretch or extend the second elastic band 28, located on the front of each of the leg openings 124 and 126. Likewise, the force needed to contract the fourth elastic band 68, located on the back of each of the pair of leg openings 124 and 126, is greater than the force needed to contract the second elastic band 28, located on the front of each of the leg openings 124 and 126.

The fourth elastic band 68 also possesses a stretching and contraction force that is greater than each of the following: that of the first elastic band 26, that of the third elastic band 66, that of the front panel 12, that of the back panel 52, and that of the waist band 128. Another way of expressing this feature is to say that in the disposable pant-like undergarment 10, the fourth elastic band 68 has the greatest stretching and contraction force.

Lastly, referring again to FIG. 2, one will notice that the second and fourth elastic bands, 28 and 68 respectively, are overlapped by the absorbent assembly 92. One will also notice that the second and fourth elastic bands, 28 and 68 respectively, are rendered non-elastic where they are overlapped by the absorbent assembly 92. The second and fourth elastic bands, 28 and 68 respectively, can be made to have a non-elastic section or portion by deactivating the elastic strands, 48 and 88 respectively, by chopping the elastic strands, by heat activating the elastic strands, by cutting the elastic strands, by chemically treating the elastic strands, or by using some other means known to those skilled in the art for destroying the stretch and contraction force of the elastic strands 48 and 88. By deactivating the elastic strands 48 and 88 overlapped by the absorbent assembly 92, one can be assured that the absorbent assembly 92 will completely contact the body of the wearer and no extend folds or pleats in the longitudinal direction will be present that could allow body fluid to leak out of the absorbent assembly 92.

While the invention has been described in conjunction with a specific embodiment, it is to be understood that many alternatives, modifications and variations will be apparent to those skilled in the art in light of the aforegoing description. Accordingly, this invention is intended to embrace all such alternatives, modifications and variations that fall within the spirit and scope of the appended claims.

1. A disposable pant-like undergarment comprising:
a) a stretchable front panel having a first end, a second end, a first side edge, a second side edge, a first elastic band secured along said first end, and a second elastic band secured along said second end;
b) a stretchable back panel having a first end, a second end, a first side edge, a second side edge, a third elastic band secured along said first end, and a fourth elastic band secured along said second end;
c) an absorbent assembly including a liquid pervious bodyside liner, a liquid-impervious outer cover, and an absorbent positioned therebetween, said absorbent assembly being secured to said stretchable front and back panels, and said absorbent assembly capable of being folded to enable said front panel to overlap said back panel; and
d) a pair of seams joining said stretchable front and back panels together at said first and second side edges to form a pant-like undergarment having a waist opening and a pair of leg openings, said first and third elastic bands creating a waist band located adjacent to said waist opening and said second and fourth elastic bands creating a pair of leg bands located adjacent to said pair of leg openings, and said fourth elastic band having a stretching force that is greater than that of said second elastic band.

2. The disposable pant-like undergarment of claim 1 having an exterior surface and an interior surface, and said waist band is located to said exterior surface and said pair of leg bands are located on said interior surface.

3. The disposable pant-like undergarment of claim 1 wherein said fourth elastic band has a contraction force that is greater than that of said second elastic band.

4. The disposable pant-like undergarment of claim 1 wherein said fourth elastic band has a stretching and contraction force that is greater than that of said first elastic band.

5. The disposable pant-like undergarment of claim 1 wherein said fourth elastic band has a stretching and contraction force that is greater than that of said third elastic band.

6. The disposable pant-like undergarment of claim 1 wherein said fourth elastic band has a stretching and contraction force that is greater than that of said front panel.

7. The disposable pant-like undergarment of claim 1 wherein said fourth elastic band has a stretching and contraction force that is greater than that of said back panel.

8. The disposable pant-like undergarment of claim 1 wherein said first elastic band extends between said first and second side edges of said front panel and said third elastic band extends between said first and second side edges of said back panel.

9. The disposable pant-like undergarment of claim 1 wherein said second and fourth elastic bands are overlapped by said absorbent assembly.

10. A disposable pant-like undergarment comprising:
a) a stretchable front panel having a first end, a second end, a first side edge, a second side edge, a first elastic band secured along said first end and extending between said first and second side edges, and a second elastic band secured along said second end and extending between said first and second side edges;
b) a stretchable back panel having a first end, a second end, a first side edge, a second side edge, a third elastic band secured along said first end and extending between said first and second side edges, and a fourth elastic band secured along said second end and extending between said first and second side edges;
c) an absorbent assembly including a liquid pervious bodyside liner, a liquid-impervious outer cover, and an absorbent positioned therebetween, said absorbent assembly being secured to said stretchable front and back panels, and said absorbent assembly capable of being folded to enable said front panel to overlap said back panel; and
d) a pair of seams joining said stretchable front and back panels together at said first and second side edges to form a pant-like undergarment having a waist opening and a pair of leg openings, said first and third elastic bands creating a waist band located adjacent to said waist opening and said second and fourth elastic bands creating a pair of leg bands located adjacent to said pair of leg openings, and said fourth elastic band having a stretching force that is at least 1.2 times greater than that of said second elastic band.

11. The disposable pant-like undergarment of claim 10 wherein said fourth elastic band has a contraction force that is greater than that of said second elastic band.

12. The disposable pant-like undergarment of claim 10 wherein said second and fourth elastic bands are overlapped by said absorbent assembly.

13. The disposable pant-like undergarment of claim 12 wherein said second and fourth elastic bands are rendered non-elastic where they are overlapped by said absorbent assembly.

14. The disposable pant-like undergarment of claim 10 wherein said waist band contains a number of elastic strands and each of said pair of leg bands contains a lesser number of elastic strands.

15. The disposable pant-like undergarment of claim 14 wherein said waist band contains the same number of elastic strands as each of said pair of leg bands.

16. A disposable pant-like undergarment comprising:
a) a stretchable front panel having a first end, a second end, a first side edge, a second side edge, a first elastic band secured along said first end and extending between said first and second side edges, and a second elastic band secured along said second end and extending between said first and second side edges;
b) a stretchable back panel having a first end, a second end, a first side edge, a second side edge, a third elastic band secured along said first end and extending between said first and second side edges, and a fourth elastic band secured along said second end and extending between said first and second side edges;
c) an absorbent assembly including a liquid pervious bodyside liner, a liquid-impervious outer cover, and an absorbent positioned therebetween, said absorbent assembly being secured to said stretchable front and back panels, and said absorbent assembly capable of being folded to enable said front panel to overlap said back panel; and
d) a pair of seams joining said stretchable front and back panels together at said first and second side edges to form a pant-like undergarment having a waist opening and a pair of leg openings, said first and third elastic bands creating a waist band adjacent to said waist opening and said second and fourth elastic bands creating a pair of leg bands adjacent to said pair of leg openings, and said fourth elastic band having a stretching force that is at least 1.5 times greater than that of said second elastic band.

17. The disposable pant-like undergarment of claim 16 wherein said fourth elastic band has a contraction force that is greater than that of said waist band.

18. The disposable pant-like undergarment of claim 16 wherein said waist band is wider than each of said pair of leg bands.

19. The disposable pant-like undergarment of claim 16 wherein said waist band has a width and each of said pair of leg bands has a width and the width of said waist band is equal to the width of each of said pair of leg bands.

20. The disposable pant-like undergarment of claim 16 wherein said waist band has a width and each of said pair of leg bands has a width and the width of said waist band is less than the width of each of said pair of leg bands.

21. The disposable pant-like undergarment of claim 16 wherein said waist band contains a number of elastic strands each of which is capable of being stretched at least 200% from an initial length and each of said pair of leg bands contains a number of elastic strands, each of which is capable of being stretched at least 200% from an initial length.
